(19) United States
(12) Patent Application Publication
(10) Pub. No.: US 2026/0248602 A1
(43) Pub. Date: Aug. 27, 2026

(54) NOSE RING APPARATUS FOR ANIMAL MONITORING

(71) Applicant: The State of Israel, Ministry of Agriculture & Rural Development, Agricultural Research Organization, Rishon-LeZion (IL)

(72) Inventors: Yael SALZER, Ometz (IL); Guy LIDOR, Rehovot (IL); Lavi ROSENFELD, Rishon Lezion (IL); Ben SHAKED, Petah Tikva (IL); Liad RESHEF, Kfar Saba (IL); Joseph GRINSHPUN, Holon (IL); Hen HONIG, Kadima-Zoran (IL); Roi SHAKED, Ein Gev (IL)

(21) Appl. No.: 18/870,805

(22) PCT Filed: May 31, 2023
(86) PCT No.: PCT/IB2023/055576

§ 371 Date: Dec. 2, 2024

Related U.S. Application Data
(60) Provisional application No. 63/347,606, filed on Jun. 1, 2022.

(51) Int. Cl. A61D 13/00; A61D 99/00

(52) U.S. Cl. CPC A61D 13/00; A61D 99/00

(57) ABSTRACT

An animal-monitoring apparatus has a housing that engages with an animal's nasal cavities via first and second arm members that each have an animal engagement portion insertable into one of the nasal cavities. A sensor arrangement is carried by the housing and has a thermal sensor and optical sensor(s). At least a portion of the thermal sensor is located in the animal engagement portion of the first or second arm member. The optical sensor(s) is/are located at a terminating end of the animal engagement portion of the first or second arm member. A processing arrangement communicates with the housing and is associated with the sensor arrangement. The processing arrangement processes signals from the thermal sensor to calculate at least one respiration-related bioparameter of the animal, and processes signals from the optical sensor(s) to calculate at least one blood circulation-related bioparameter of the animal.

### CROSS-REFERENCE TO RELATED APPLICATIONS

This application claims priority from U.S. Provisional Patent Application No. 63/347,606, filed Jun. 1, 2022, the disclosure of which is incorporated by reference in its entirety herein.

### TECHNICAL FIELD

The present disclosure relates to monitoring animals, and in particular monitoring animal bioparameters using a nose ring that carries one or more sensors.

### BACKGROUND OF THE INVENTION

Monitoring of cardiovascular and respiratory measurements in animals, such as livestock animals, can be used to monitor and assess the welfare and health of an animal. Changes in pulse rate, breathing rate, and oxygen saturation are valuable metrics in veterinary medicine that are used to assess stress, pain, illness, and detect critical conditions. For monitoring pulse rate and oxygen saturation in animals, conventional techniques utilize hand-held pulse oximetry devices. However, using hand-held pulse oximetry devices requires labor-intensive human interaction with the animals, and cannot provide continuous pulse and saturation information. For monitoring breathing rate, conventional techniques rely on human counting of flank movements in the animal. These techniques are time-consuming and labor-intensive and require training, and are prone to human error.

### SUMMARY OF THE INVENTION

The present disclosed subject matter, also referred to herein as the disclosure, includes an apparatus, device, system, and method for monitoring an animal.

According to the teachings of an embodiment of the present disclosure, there is provided an apparatus for monitoring an animal. The apparatus comprises: a housing configured to engage with nasal cavities of the animal, the housing including a first arm member and a second arm member, each of the arm members having an animal engagement portion configured to be inserted into one of the nasal cavities; a sensor arrangement carried by the housing and including: a thermal sensor having at least a portion thereof located in the animal engagement portion of the first or second arm member, the thermal sensor for measuring nasal cavity air temperature, and at least one optical sensor located at a terminating end of the animal engagement portion of the first or second arm member, the at least one optical sensor for sensing light from the septum of the animal; and a processing arrangement in communication with the housing and electrically associated with the sensor arrangement, the processing arrangement configured to: process signals from the thermal sensor to calculate at least one respiration-related bioparameter of the animal, and process signals from the at least one optical sensor to calculate at least one blood circulation-related bioparameter of the animal. Optionally, the at least one optical sensor senses light, produced by at least one light source, that is reflected from the septum.

Optionally, the at least one optical sensor senses light, produced by at least one light source, that is transmitted by the septum.

Optionally, the apparatus further comprises: an illumination arrangement including at least one light source for producing the light from the septum of the animal that is sensed by the at least one optical sensor, the illumination arrangement located at a terminating end of the animal engagement portion of the first arm member.

Optionally, the apparatus further comprises: an illumination arrangement including at least one light source for producing the light from the septum of the animal that is sensed by the at least one optical sensor, the illumination arrangement located at a terminating end of the animal engagement portion of the second arm member.

Optionally, the at least one optical sensor includes a plurality of optical sensors, each of the optical sensors being sensitive to a different respective wavelength.

Optionally, the thermal sensor and the at least one optical sensor are located in the same arm member.

Optionally, the thermal sensor and the at least one optical sensor are located in different arm members.

Optionally, the first or second arm member has a hollow interior section forming a channel in at least part of the animal engagement portion, an aperture in a major external surface of the animal engagement portion communicates with the channel, and a portion of the thermal sensor is located in the channel and another portion of the thermal sensor protrudes from the aperture.

Optionally, the first or second arm member has a hollow interior section forming a channel in at least part of the animal engagement portion and at least part of a central portion of the first or second arm member, and at least part of one or more wired transmission medium that carry signal from one or more sensor of the sensor arrangement to the processing arrangement is located in the channel.

Optionally, each of the animal engagement portions includes a terminating end, and one of the terminating ends is circular shaped to reduce discomfort to the animal.

Optionally, each of the animal engagement portions includes a terminating end, and one of the terminating ends has a configuration that accommodates an optical module having the at least one optical sensor and that reduces discomfort to the animal.

Optionally, each of the animal engagement portions includes a terminating end, and the housing includes an adjustment mechanism configured to adjust a lateral spacing between the terminating ends.

Optionally, the housing further including a base member, and each of the arm members includes a base engagement portion that mechanically couples the arm member to the base member, at least one of the base engagement portions configured to slide along the base so as to adjust a lateral spacing between the terminating ends.

Optionally, the processing arrangement includes at least one first processor for processing signals from the thermal sensor and at least one second processor for processing signals from the at least one optical sensor.

Optionally, at least one processor of the processing arrangement is carried by the housing.

Optionally, the processing arrangement is internal to the housing.

Optionally, further comprising: a communication module configured to send one or more of the calculated bioparameters to a computing device that is remote from the apparatus. Optionally, the communication module is further configured to send to the computing device raw sensor measurements from one or more of the thermal sensor or the at least one optical sensor, and the computing device is configured to process the raw sensor measurement to extract information associated with the animal including at least diagnostic information.

There is also provided according to an embodiment of the teachings of the present disclosure an apparatus for monitoring an animal. The apparatus comprises: a housing configured to engage with the nasal cavities of the animal, the housing including: a first arm member and a second arm member, each of the arm members having an animal engagement portion configured to be inserted into one of the nasal cavities, each of the animal engagement portions including a terminating end, and an adjustment mechanism configured to adjust a lateral spacing between the terminating ends; and a sensor arrangement carried by the housing and including one or more sensor located in one of the animal engagement portions and operative to collect sensor measurements from one of the nasal cavities indicative of one or bioparameter of the animal.

Optionally, the apparatus further comprises: a processing arrangement carried by the housing, the processing arrangement including at least one hardware processor in communication with at least one storage medium and configured to process signals from the one or more sensor to derive the one or more bioparameter.

There is also provided according to an embodiment of the teachings of the present disclosure a system for monitoring an animal. The system comprises: an animal monitoring apparatus including: a housing configured to engage with the nasal cavities of the animal, the housing including a first arm member and a second arm member, each of the arm members having an animal engagement portion configured to be inserted into one of the nasal cavities, a sensor arrangement carried by the housing and including: a thermal sensor having at least a portion thereof located in the animal engagement portion of the first or second arm member, the thermal sensor for measuring nasal cavity air temperature, and at least one optical sensor located at a terminating end of the animal engagement portion of the first or second arm member, the at least one optical sensor for sensing light from the septum of the animal, and a communications module carried by the housing; a processing subsystem in communication with the sensor arrangement and configured to: process signals from the thermal sensor to calculate at least one respiration-related bioparameter of the animal, and process signals from the at least one optical sensor to calculate at least one blood circulation-related bioparameter of the animal; and a computing device that is in communication with the animal monitoring apparatus via one or more wireless communication network, the communications module is configured to send to the computing device, over the one or more wireless communication network, raw sensor measurements from the sensor arrangement, and the computing device is configured to process the raw sensor measurement to extract information associated with the animal including at least diagnostic information.

Optionally, the animal monitoring apparatus includes the processing subsystem, and the processing subsystem is carried by the housing.

Within the context of this document, the term “animal” refers to non-human mammalian animals, in particular, livestock such as cattle (both dairy cattle and beef cattle) sheep, goats, and pigs, but also includes other animals including other ruminants, boars, as well as work animals such those belonging to the Equidae family (e.g., horses, donkeys, etc.), zoo animals, and the like.

Unless otherwise defined herein, all technical and/or scientific terms used herein have the same meaning as commonly understood by one of ordinary skill in the art to which the disclosure pertains. Although methods and materials similar or equivalent to those described herein may be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the present disclosure are herein described, by way of example only, with reference to the accompanying drawings. With specific reference to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

Attention is now directed to the drawings, where like reference numerals or characters indicate corresponding or like components. In the drawings:

FIG. 1 is an isometric view of an apparatus, according to embodiments of the present disclosure, having a housing with a base member connected to arm members having animal engagement portions for engaging with the nasal cavities of an animal, and a sensor arrangement having thermal and optical sensors in one of the animal engagement portions for collecting sensor measurements indicative of bioparameters of the animal;

FIG. 2 is an isometric view showing the apparatus of FIG. 1 from a different perspective;

FIG. 3 is an isometric view showing one of the arm members of the apparatus of FIG. 1 from a different perspective, and without the thermal sensor;

FIG. 4 is a central-plane cross-sectional view of the apparatus of FIG. 1;

FIG. 5 is an isometric exploded view of a terminating region of one of the engagement members of the apparatus of FIG. 1, illustrating a cover member and an optical module that carries the optical sensors of the sensor arrangement positioned relative to an opening of the terminating region;

FIG. 6 is an isometric view showing the apparatus of FIG. 1 from a different perspective and without the base member of the housing;

FIG. 7 is an isometric view similar to FIG. 6 but from a different perspective;

FIG. 8 is an isometric view illustrating a schematic representation of the apparatus of FIG. 1 engaged with the nose of an animal;

FIG. 9 is a schematic block diagram showing electronic and optical components of the apparatus, according to embodiments of the present disclosure; and

FIG. 10 is a diagram illustrating a system having the apparatus of FIG. 1 and a computing device, according to embodiments of the present disclosure, and a system environment in which embodiments of the present disclosure can be deployed.

### DESCRIPTION OF THE PREFERRED EMBODIMENTS

The present disclosure includes an apparatus, device, system, and method for monitoring an animal.

The principles and operation of the apparatus, device system, and method according to present disclosure may be better understood with reference to the drawings accompanying the description.

Embodiments of the present disclosure are applicable for use in any situation in which non-invasive health and/or behavior monitoring of an animal is desired, and is of particular value when applied to monitoring of livestock and even more particularly dairy cattle and beef cattle. It is noted, however, that the embodiments of the present disclosure are also suitable for monitoring the health and/or behavior of other animals, including, but not limited to, sheep, goats, pigs, ruminants (aside from cattle), boars, as well as work animals such those belonging to the Equidae family (e.g., horses, donkeys, etc.), and zoo animals.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the examples. The disclosure is capable of other embodiments or of being practiced or carried out in various ways. Initially, throughout this document, references are made to directions, such as, for example, inner and outer, and the like. These directional references are exemplary only to illustrate the disclosure and the embodiments thereof.

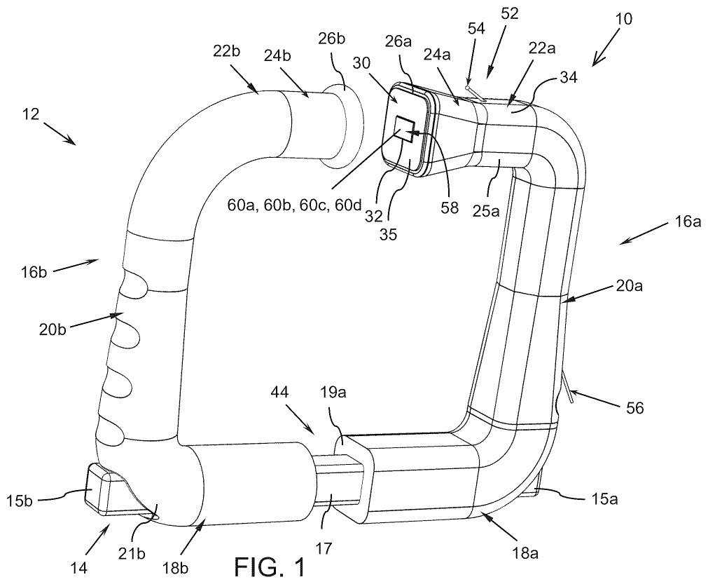

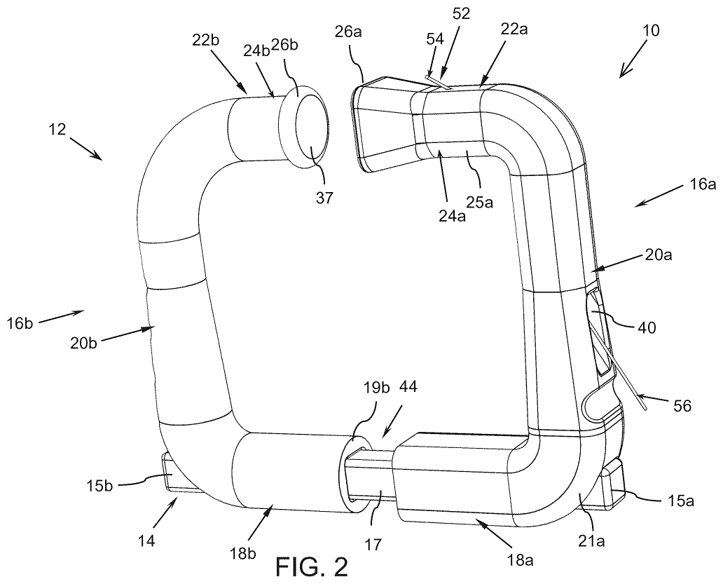

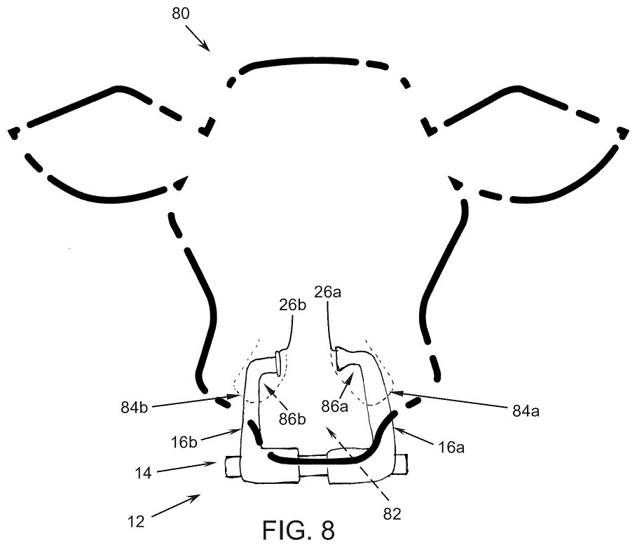

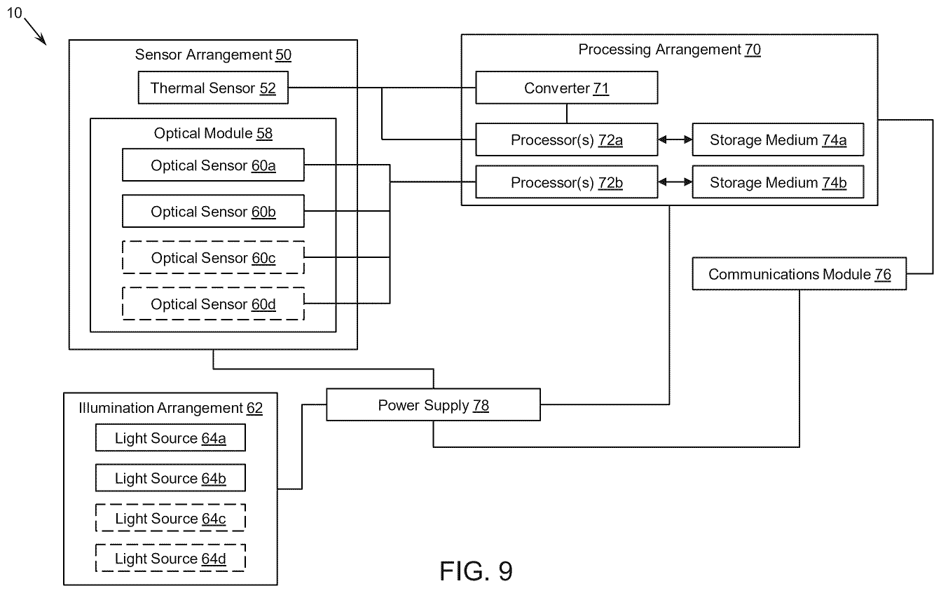

Referring now to the drawings, FIGS. 1-10 illustrate various views and components of an apparatus, generally designated 10, constructed and operative according to a non-limiting embodiment of the present disclosure, for monitoring an animal. Generally speaking, the apparatus 10 includes a housing 12 that is configured to securely engage with the nasal cavities 86a, 86b of the animal (FIG. 8), a sensor arrangement 50 (FIG. 9) that is carried by the housing 12 and includes thermal and optical sensors 52, 60a, 60b, 60c, 60d configured to collect measurements from the nasal cavities that are indicative of one or more bioparameter of the animal, and a processing arrangement (also referred to interchangeably herein as a “processing subsystem”) 70 (FIG. 9) that is connected to the housing 12 (in certain embodiments carried by the housing 12) and is configured to process signals from sensors of the sensor arrangement 50 to calculate/derive the one or more bioparameter. The one or more bioparameter can include one or more of respiration-related bioparameters of the animal such as breathing rate (also referred to as “respiration rate”) of the animal, breathing rate variability (BRV), and breath intake/volume, and blood circulation-related bioparameters of the animal such as pulse rate of the animal, heart rate variability (HRV), blood volume and/or pressure, and the peripheral oxygen saturation (SpO₂) level of the animal. Other informative biometrics may also be derived from the sensor signals/measurements. At least some of the aforementioned bioparameters can be calculated/derived from measurement, by one or more of the sensors of the sensor arrangement 50, of direct or raw sinusoidal breathing signals and/or sinusoidal pulse signals. As is known in the art, the oxygen saturation level is the fraction of oxygen-saturated hemoglobin relative to total hemoglobin in the blood of the animal.

As will be discussed, the housing 12, which is configured as a nose ring, and the location of the sensors of the sensor arrangement 50 in the nose ring housing 12, are designed according to an “animal-centered design” paradigm such that the apparatus 10 is able to accurately measure the aforementioned bioparameters while at the same time providing minimal discomfort to the animal.

The housing 12 may be constructed from plastic or other suitable material having a suitable degree of flexibility that is ideal for use in animal nose rings.

With particular reference to FIGS. 1-4 and FIG. 8, the housing 12 has a pair of arm members (also referred to as “prongs” or “branches”) 16a and 16b that have animal engagements portions 22a and 22b that are configured to be inserted into different respective nasal cavities 86a and 86b of the animal 80 through respective nostrils 84a and 84b of the animal so as to engage the housing 12 within the nasal cavities 86a, 86b of the animal 80. The arm members 16a and 16b also have central portions 20a and 20b, which can be elongated portions. In the illustrated embodiment, the arm members 16a and 16b also have base engagement portions 18a and 18b for mechanically attaching the arm members 16a and 16b to a base member 14 of the housing 12. The mechanical attachment will be discussed in further detail in subsequent sections of this document.

In the illustrated embodiment, the animal engagement portions 22a and 22b project inward such that the housing 12 has a general U-shape with inwardly projecting ends. In certain embodiments, the housing may not include a base member, and the central portions 20a and 20b may be attached to each other at a common end region of the central portions 20a and 20b to form a V-shape or Y-shape. It should be understood that the housing of the apparatus according to embodiments of the present disclosure can take various forms beside the U-shape illustrated in the drawings, and the V-shapes or Y-shape mentioned above. The housing, in principle, can be of any other shape that has two arms or branches, that have animal engagement portions, and that are attached to each other in some way and that can be used in an animal nose ring configuration.

The animal engagement portion 22a forms a continuation of the central portion 20a resulting in a smooth transition between the portions 20a and 22a. The base engagement portion 18a also forms a continuation of the central portion 20a resulting in a smooth transition between the portions 20a and 18a. The smooth transition regions are in the form of bends, for example of approximately 90 degrees.

The animal engagement portion 22b forms a continuation of the central portion 20b resulting in a smooth transition between the portions 20b and 22b. The base engagement portion 18b also forms a continuation of the central portion 20b resulting in a smooth transition between the portions 20b and 18b. Here too the smooth transition regions are in the form of bends, for example of approximately 90 degrees.

In certain embodiments, the portions 18a, 20a, 22a of the arm member 16a form a single unitary piece, as do the portions 18b, 20b, 22b of the arm member 16b. Construction of the arm members 16a and 16b as single unitary pieces can be achieved, for example, by manufacturing each of the arm members 16 a and 16 b as a single piece using, for example, 3D printing, molding (e.g., injection molding), or any other suitable technique. Alternatively, the portions of each of the arm members can be separately manufactured and then attached to each other using adhesive bonding or other suitable bonding techniques.

The animal engagements portions 22a and 22b have terminating regions 24a and 24b, which are the regions (or portions) of the animal engagements portions 22a and 22b that are positioned closest to the septum of the animal when the housing 12 is engaged with the nose 82 of the animal 80 (FIG. 8). The terminating regions 24a and 24b have terminating ends 26a and 26b, at least one of which carries the optical sensor(s) 60a, 60b, 60c, 60d. The terminating end 26a or 26b that carries the optical sensor(s) 60a, 60b, 60c, 60d is in direct contact with the septum of the animal when the housing 12 is engaged with the nose of the animal, in order to obtain optical signal readings of sufficient signal strength. The other terminating end 26b or 26a, i.e., the terminating end without the optical sensor(s), is preferably positioned in close proximity to the septum of the animal, preferably within a few millimeters, e.g., within 5 millimeters, of the septum, when the housing 12 is engaged with the nose of the animal.

In accordance with the animal-centered design paradigm, a majority portion of animal engagement portions 22a and 22b have shape that reduce discomfort to the animal. In the non-limiting embodiments illustrated in the drawings, a majority portion of the animal engagement portion 22a has a rectangular cross-section, and a majority portion of the animal engagement portion 22b is cylindrical in shape (having has a circular cross-section).

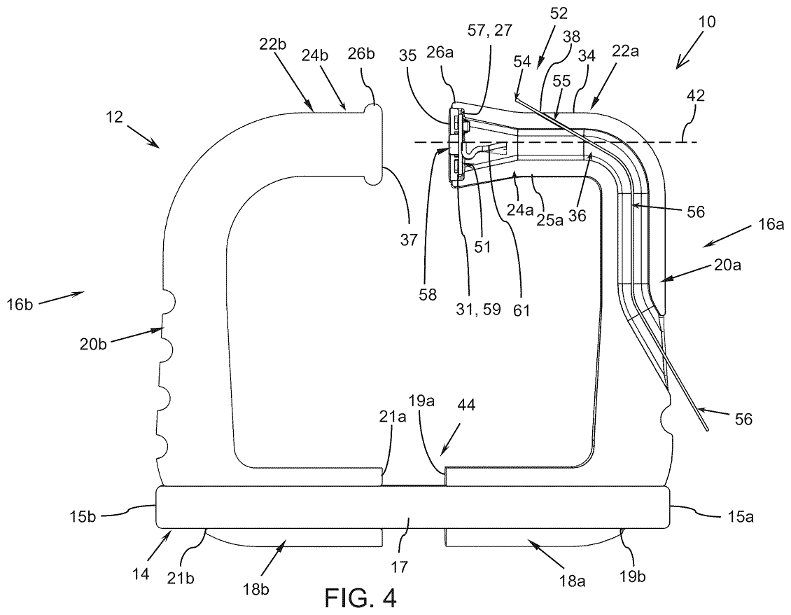

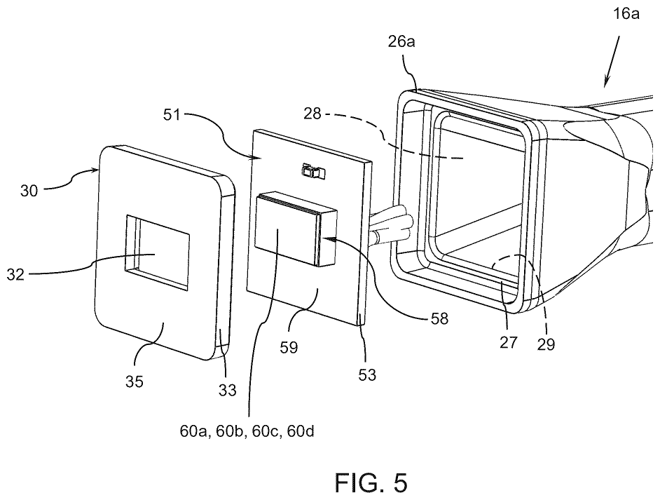

In certain embodiments, each of the terminating ends 26a and 26b has a configuration (i.e., design) that reduces discomfort to the animal, in accordance with the animal-centered design of the housing 12. This configuration can include, for example, smooth or rounded edges or outer surfaces, being of a circular or oblong shape, being of a rectangular shape with rounded corners, and the like. As illustrated in FIGS. 4 and 5, the configuration of the terminating end 26a (or the terminating end 26b) is also such that it accommodates an optical module 58 that carries the optical sensors 60a, 60b, 60c, 60d of the sensor arrangement 50. The optical module and optical sensors are represented schematically in the drawings as a rectangular cuboid. The optical module 58 can include a transparent window (not shown) that covers and protects the optical sensors, and is transparent to light to light at wavelengths to which the optical sensors are sensitive. As shown in FIG. 5, the optical module 58 can be mounted to a planar substrate 51 that has an inner surface 57, an outer surface 59 opposite the inner surface 57, and a peripheral side surface 53. The substrate can also carry circuitry or wiring that carries signal from the optical sensors to the processing arrangement 70.

In the non-limiting example embodiment illustrated in the drawings, the configuration of the terminating end 26b is such that the terminating end 26b is circular shaped, and the configuration of the terminating end 26a is such that the terminating end 26a is rectangular-shaped (or quasi-rectangular-shaped). As illustrated in FIGS. 4 and 5, the rectangular-shaped configuration also accommodates a rectangular-shaped substrate 51.

With continued reference to FIG. 5, in certain embodiments an opening 28 is formed at the terminating end 26a. The opening 28 is configured in size/dimension to receive the substrate 51 and the optical module 58. Although the substrate 51 shown in the drawings has a rectangular footprint, it should be appreciated that the substrate 51 may be designed with a circular or oblong footprint that is conducive to being received in a circular or oblong terminating end 26a, which can even further reduce discomfort to the animal.

In certain embodiments, the terminating end 26b has the form of a non-flat disk having a planar outer surface 37 that defines the circular shape of the terminating end 26b. The diameter of the disk is slightly larger than the diameter of remaining cylindrical portion of the animal engagement portion 22b. This slight increase in diameter can help maintain the animal engagement portion 22b in the nasal cavity of the animal.

With continued reference to FIG. 4, in certain embodiments the arm member 16a has a hollow interior section that forms a channel 36 in at least part of the animal engagement portion 22a such that the channel 36 extends through at least part of the animal engagement portion 22a. The channel 36 may also extend through the bend between the animal engagement portion 22a and the central portion 20a and into at least part of the central portion 20a. In certain embodiments, the opening 28 formed at the terminating end 26a is also part of the channel 36. As will be discussed, the channel 36 can be used to support one or more wired transmission medium that carry signal from one or more sensors of the sensor arrangement 50 to the processing arrangement 70. For example, wire(s) 61 that carry signal from the optical sensors can be located in the channel 36. One or more of the wire(s) 61 may also be used to provide power to the optical sensors from a power supply (for example the power supply 78 in FIG. 9).

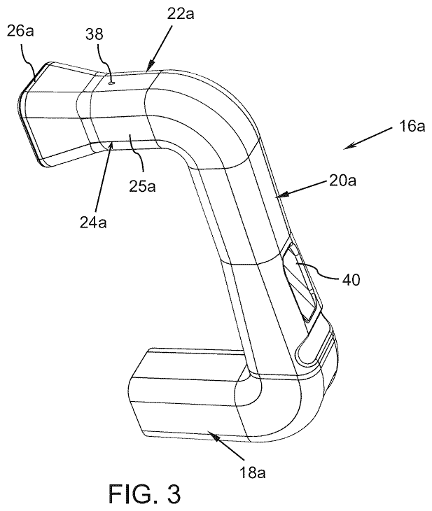

In certain embodiments, for example as illustrated in FIGS. 3 and 4, an aperture 38 is formed in a portion of the major external surface 34 of the arm member 16a and communicates with the channel 36. As will be discussed, the portion of the major external surface 34 at which the aperture 38 is formed is in the animal engagement portion 22a (which may, but not necessarily, be in the terminating region 24a) in order to accommodate a thermal sensor 52 of the sensor arrangement 50 to protrude out from the housing 12. The aperture 38 extends through the interior of the animal engagement portion 22a to reach the channel 36. There may also be an aperture or opening 40 formed in another portion of the major external surface 34 (in the central portion 20a) that communicates with the channel 36 for accommodating one or more wired transmission medium, that carry signal from one or more sensors of the sensor arrangement 50 to the processing arrangement 70, to exit the housing 12. As will be discussed, there are embodiments contemplated in which the apparatus 10 is an untethered device in which all electronic and processing components are internal to the housing 12, thereby rendering moot the need for the opening 40.

In certain embodiments, the terminating end 26a also includes a cover member 30 which is configured to engage with the terminating end 26a so as to cover the opening 28 (and the optical module 58 that is in the opening 28). As illustrated in FIGS. 4 and 5, a support rim 27 may be formed on a portion of the interior surface 29 of the terminating region 24a that is close to the terminating end 26a. The support rim 27 extends along the portion of the interior surface 29, and projects inward from the interior surface 29 by a small amount (where a “small amount” here can be an order of magnitude less than the diameter of the terminating end 26a). When the optical module 58 is mounted in the opening 28, peripheral portions of the inner surface 57 of the substrate 51 rest on the support rim 27 (FIG. 4), and the side (peripheral) surface 53 of the substrate 51 abuts a portion of the interior surface 29. When the cover member 30 covers the opening 28 and the optical module 58, the inner surface 31 is in close contact with the outer surface 59 of the substrate 51, and the side (peripheral) surface 33 of the cover member 30 abuts a portion of the interior surface 29.

The cover member 30 and the terminating end 26a may be correspondingly configured in size/dimension such that the cover member 30 can be snapped into place to cover the opening 28 and can also be pried out of (removed from) the opening 28 (to enable access to the optical module 58). In other embodiments, the cover member 30 may be adhesively bonded to the opening 28 to seal the opening 28 more effectively.

At least a portion of the cover member 30 is preferably transparent for light at wavelengths to which the optical sensors 60a, 60b, 60c, 60d are sensitive. In other words, at least a portion of the cover member 30 transmits light at wavelengths to which the optical sensors are sensitive. In the non-limiting embodiment illustrated in the drawings, the transparent portion of the cover member 30 is in the form of an aperture 32 that extends between the inner and outer surfaces 31 and 35 of the cover member 30, and that occupies a portion of the surface area of the cover member 32. The aperture 32 and the portion of the optical module 58 that carries the optical sensors are preferably correspondingly configured in size/dimension such that the optical module 58 and optical sensors can be received in the aperture 32. In addition, the aperture 32 is preferably large enough to accommodate the field of view of the optical sensors.

With continued reference to FIGS. 1-5, in certain embodiments the terminating region 24a expands radially outward when moving along a central axis 42 of the terminating region 24a from a beginning portion 25a of the terminating region 24a toward the terminating end 26a. The central axis 42 is perpendicular to a planar outer (exterior) surface 35 of the cover member 30. The outer surface 35 is generally opposite the inner surface 31, and is in facing relation to the outer surface 37 of the terminating end 26b. The outer surfaces 35 and 37 are also preferably parallel or approximately parallel to each other. The radial expansion when moving along the central axis 42 is preferably gradual, such that the diameter of terminating region 24a gradually changes between the beginning portion 25a and the terminating end 26a. The radial expansion results in the terminating region 24a being “flared-out” such that the terminating region 24a has the shape of a conical frustum in two cross-sections (the cross-section shown in FIG. 4 which is taken along one of the two planes that is perpendicular to the surface 35 of the cover member 30, and the cross-section taken along the other of the two planes that is perpendicular to the planar surface 35 of the cover member 30). This flared-out configuration is also in accordance with the animal-centered design of the housing 12, reducing the overall discomfort to the animal when the housing 12 is engaged with the nose of the animal.

Although the embodiments described thus far have pertained to one of the terminating ends 26a being quasi-rectangular-shaped (with rounded corners) and the other of the terminating ends 26b being circular (non-flat disk) shaped, embodiments are contemplated herein in which both terminating ends are circular shaped, both terminating ends are quasi-rectangular-shaped, or one or both of the terminating ends is of another geometric shape (albeit that some of such other geometric shapes may, to some degree, increase discomfort to the animal).

In certain embodiments, the lateral spacing between the terminating ends 26a and 26b (which can be defined by the lateral spacing between the outer surfaces 35 and 37) is fixed. However, this fixed spacing may be less ideal, as adjustment to fit different sizes of nasal features (e.g., different septum thickness) and different animal types may be limited to the natural flexure and resilient bias of the material or materials from which the arm members 16a and 16b are constructed. It is therefore a particular feature of certain aspects of the present disclosure, in accordance with the animal-centered design paradigm, to provide an adjustment mechanism that can adjust the lateral spacing between the terminating ends 26a and 26b to better fit the housing 12 to the nose of the animal with minimal discomfort to the animal.

With particular reference to FIGS. 1, 2, and 4, certain non-limiting embodiments of the present disclosure show the housing 12 having an adjustment mechanism 44 that is configured to adjust the lateral spacing between the terminating ends 26a and 26b. In practice, the spacing adjustment can be effectuated by the adjustment mechanism 44 laterally moving the arm members 16a and 16b one relative to the other. In certain embodiments, one of the arm members 16a or 16b can be fixed in place, and the adjustment mechanism 44 can move the other of the arm members 16b or 16a laterally relative to the arm member 16a or 16b. In other embodiments, both of the arm members 16a and 16b are laterally moveable.

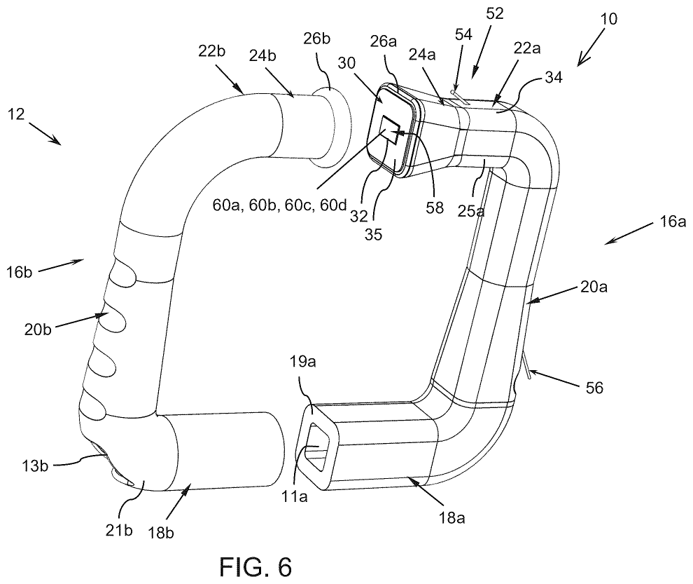

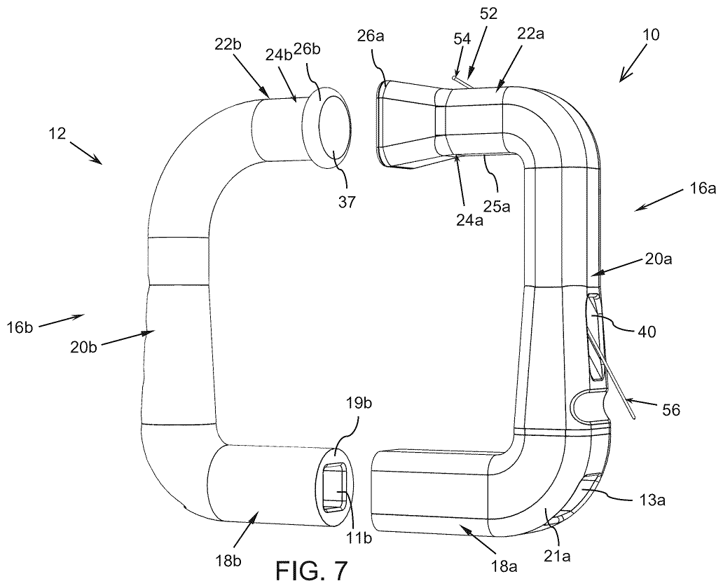

In one non-limiting embodiment, such as the embodiment illustrated in FIGS. 1, 2, and 4, the adjustment mechanism 44 is implemented as a slider mechanism. Here, the arm members 16a and 16b are configured to slide along the base member 14 between opposite terminating ends 15a and 15b of the base member 14. The sliding engagement is enabled by providing the base engagement portions 18a and 18b with hollow interior portions that are conformed to the shape of the base member 14 (i.e., the hollow interior portions and the base member are “correspondingly configured”) and that provide a mechanical coupling between the arm members 16a and 16b and the base member 14. The hollow interior portions of the base engagement portions 18a and 18b extend through the entirety of the base engagement portions 18a and 18b. In particular the hollow interior portion of the base engagement portion 18a extends through a terminating end 19a and an end region 21a that is near where the base engagement portion 18a forms a continuation of the central portion 20a, and the hollow interior portion of the base engagement portion 18b extends through a terminating end 19b (that is in facing relation to the terminating end 19a) and an end region 21b that is near where the base engagement portion 18b forms a continuation of the central portion 20b. FIGS. 6 and 7 show the hollow openings 11a and 11b in the terminating ends 19a and 19b and the hollow openings 13a and 13b in the end regions 21a and 21b. The base member 14 extends through the hollow base engagement portions 18a and 18b (i.e., through the openings 13a, 11a, 11b, 13b), thereby allowing the base engagement portions 18a and 18b to slide along the base member 14.

A locking mechanism, not shown, can be deployed to lock the arm members 16a and 16b in position one relative to the other in order to fix the lateral spacing between the terminating ends 26a and 26b. In one embodiment, the locking mechanism can be implemented as one or more rubber stopper or clamp placed at either or both of the terminating ends of the base member 15. In other embodiments, threading engagement can be used to adjust the lateral spacing. For example, one or more portions of the base member 14, for example the terminating ends 15a and 15b and the central portion 17 of the base member 14, can have threading, and fasteners (e.g., a nut) having threading configured to engage with the threaded portions of the base member 14 can be used to lock the either or both of the arm members 16a and 16b in place. It is noted that when using threaded engagement, the base member 14 should be cylindrical in shape, and not cuboid in shape as illustrated in the drawings. Moreover, the hollow interior portions of the base engagement portions 18a and 18b should also be cylindrical to conform to the shape of the base member.

Threading can also be employed to implement the adjustment mechanism 44. For example, in certain embodiments, the base member 14 can have threading along the entirety or majority of its entire length, and the hollow interior portion of one of the base engagement portions 18a or 18b can have corresponding threading such that rotation of the base member 14 along its long central axis causes the base engagement portion 18a or 18b to move laterally along the base member 14b.

Implementing the adjustment mechanism 44 as a slider mechanism or a threaded engagement mechanism has advantages that the lateral spacing can be adjusted while the housing 12 is already engaged with the nose of the animal. For example, if the lateral spacing is such that the housing 12 is somewhat loose on the animal or the terminating ends 26a and 26b are too far from the septum, resulting in poor sensor measurements, the adjustment mechanism 44 can be used to tighten the housing 12 to better fit on the nose of the animal without removing the housing 12 from the animal. Nevertheless, other implementations of the adjustment mechanism 44 that require disengagement from the animal are contemplated herein. In one non-limiting embodiment, spacers can be used to adjust the lateral spacing between the terminating ends 26a and 26b. For example, the housing 12 can be initially mounted on the animal to assess the required spacing to securely attach the housing 12 to the animal. The housing 12 can then be removed from the animal and an appropriate number of spacers can be threaded onto the base member 14 between the base engagement portions 18a and 18b to achieve the desired lateral spacing between the terminating ends 26a and 26b.

It is noted that in the embodiments illustrated in the drawings, the base engagement portions 18a and 18b are elongated portions that project inward, such that terminating ends 19a and 19b can be brought into relatively close proximity to each other. This construction may provide additional stability to the housing 12, particularly in embodiments in which the adjustment mechanism 44 is implemented as a slider mechanism (as discussed above). It is noted, however, that embodiments are contemplated herein in which the base engagement portions 18a and 18b have little to no elongation such that when the housing 12 is maximally expanded (i.e., the lateral spacing between the terminating ends 26a and 26b is maximal) the terminating ends 19a and 19b are close to the terminating ends 15a and 15b (and central portions 20a and 20b), respectively.

With reference to FIGS. 1, 2, and 4-7, the following paragraphs describe some of the electronic and optical components of the apparatus 10, including the components of the sensor arrangement 50 and the processing arrangement 70, and the functions performed by those components.

The sensor arrangement 50 preferably includes the thermal sensor 52 that is operative to measure the air temperature in the nasal cavity of the animal. The thermal sensor 52 is located in one of the animal engagement portions 22a or 22b (portion 22a in the example embodiment illustrated in the drawings). The location of the thermal sensor 52 may or may not be in one of the terminating regions 24a or 24b. The thermal sensor 52 is deployed in the animal engagement portion 22a or 22b such that the thermal sensor 52 is located within the nasal cavity but is not in contact with the skin within the nasal cavity when the housing 12 is engaged with the nose of the animal. In the illustrated embodiments, a single thermal sensor 52 is deployed such that air temperature measurements are obtained from a single nasal cavity 86a. It is noted, however, that multiple thermal sensors can be used. For example, multiple thermal sensors can be deployed in a single one of the arm members to obtain multiple sets of thermal sensor measurements from a single nasal cavity 86a, or one or more thermal sensor can be deployed in each of the arms 16a and 16b such that air temperature measurements can be obtained from both of the nasal cavities.

The thermal sensor 52 can be implemented in various ways, for example as a probe type sensor, such as a type-K thermocouple. The thermal sensor 52 has a body 55 that terminates in a measurement portion 54, for example in the form of a probe tip, that protrudes out form the aperture 38 such that the measurement portion 54 is exposed. The remaining portions of the probe body 55 are retained within the channel 36. In the illustrated embodiment, the thermal sensor 52 also includes a wire or wires (wired transmission medium) 56, the majority portion of which is/are located in the channel 38, and carry temperature-indicative signal from the body 55 to the processing arrangement 70.

In certain embodiments, the processing arrangement 70 includes processing elements that include one or more computerized (hardware) processor 72a which operate to process the raw temperature-indicative signal received from the thermal sensor 52 to calculate respiration-related bioparameters of the animal, including the breathing rate of the animal, the animal's breath intake/volume, and BRV. A thermocouple-to-digital converter 71 can also be provided to enhance the temperature-indicative signal prior to the signal reaching the process(s) 72a. The temperature-indicative signal is an oscillating signal that oscillates with the nasal cavity inhale-exhale activity. The processor(s) 72a may sample the nasal cavity inhale-exhale oscillating signal (the temperature-indicative signal) at an appropriate sample rate (e.g., 30 Hz which meets the minimum Nyquist rate of 5 Hz for a speeded heart rate of 150 beats per minute) to calculate the breathing rate of the animal by tracking the nasal cavity inhale-exhale alternations, for example by performing a signal peak count (e.g., calculating the times between peaks and valleys in the inhale-exhale oscillating signal). The processor(s) 72a may also calculate breathing rate by performing Fourier analysis, for example Fast Fourier Transform (FFT), on the center frequency of the temperature-indicative signal output by the thermal sensor 52. The processor(s) 72a may calculate breath intake/volume for example by multiplying the raw signal pulse width by its amplitude. The processor(s) 72a may calculate BRV using various techniques, including time-domain analysis and frequency-domain analysis, as in known in the art.

It is noted that in effect, the raw measurements of thermal sensor 52 can be logged and stored, regardless of the signal processing techniques applied to the signals by the processing arrangement 70.

The sensor arrangement 50 also preferably includes the one or more optical sensor 60a, 60b, 60c, 60d for sensing light from the septum of the animal. The processing arrangement 70 includes one or more processor 72b that operates to process signals from the optical sensors to calculate blood circulation-related bioparameters of the animal, including pulse rate of the animal, oxygen saturation level (SpO₂) of the animal, blood volume and/or pressure, and HRV.

The optical sensors 60a, 60b, 60c, 60d are preferably carried by the optical module 58 that is accommodated in the opening 28 formed at the terminating end 26a. The optical sensors 60a, 60b, 60c, 60d may be photoplethysmography (PPG) sensors (photodetectors, e.g., photodiodes) which are commonly used in pulse oximetry to determine pulse rate and SpO₂. Although four optical sensors are shown in FIG. 9, any suitable number of optical sensors can be used, including a single optical sensor, two optical sensors, and so on.

The processing arrangement 70, for example the processor(s) 72b, may sample the pulse rate signal (extracted from the signals produced by the optical sensors) at an appropriate sample rate (e.g., 30 Hz which meets the minimum Nyquist rate of 5 Hz for a speeded heart rate of 150 beats per minute) to calculate the pulse rate of the animal by tracking the pulse rate signal alternations, for example by performing a signal peak count (e.g., calculating the times between peaks and valleys in the pulse rate oscillating signal). The processing arrangement 70, for example the processor(s) 72b, may also calculate pulse rate by counting the number of peaks in the raw signal output by the optical sensors. The processing arrangement 70, for example the processor(s) 72b, may also calculate pulse rate by performing Fourier analysis, for example FFT, on the center frequency of the raw signal output by the optical sensors. The processing arrangement 70, for example the processor(s) 72b, may calculate HRV by, for example, calculating the changes in the time-intervals between consecutive heartbeats (inter-beat intervals) from the raw pulse rate signal. Other techniques for calculating HRV are well-known in the art.

It is noted that in effect, the raw measurements of optical sensors 60a, 60b, 60c, 60d can be logged and stored, regardless of the signal processing techniques applied to the signals by the processing arrangement 70.

In a first set of embodiments, the optical sensors 60a, 60b, 60c, 60d detect light reflected from the septum of the animal. In a second set of embodiments, the optical sensors 60a, 60b, 60c, 60d detect light that is transmitted through the septum of the animal. In both sets of the embodiments, the light that is detected by the optical sensors 60a, 60b, 60c, 60d originates from an illumination arrangement 62 that has one or more light source 64a, 64b, 64c, 64d. Each light source 64a, 64b, 64c, 64d can be implemented as any suitable light source, but implementation of the light sources 64a, 64b, 64c, 64d as light-emitting diodes (LEDs) has been found to be an effective implementation. Although not shown in the drawings, the illumination arrangement 62 may also include illumination drivers for regulating power to the light sources. Parenthetically, the illumination drivers can receive power from a power supply 78 (FIG. 9) of the apparatus 10, which can, in certain embodiments, provide power to all of the components of the apparatus 10 that require power, including the sensors of the sensor arrangement 50 (if such sensors require external power), the processing arrangement 70, and a communications module 76 (FIG. 9). The power supply 78 can, for example, be a battery power supply, such as a 3.7-volt battery.

It has been found that certain wavelengths are particularly suitable for pulse oximetry, and that the septum of certain animals, in particular cattle, reflect light and also transmit light at those certain wavelengths with enough efficiency that the reflected/transmitted light can be picked up by the optical sensors. The wavelengths that have been found to be particularly suitable for pulse oximetry are in a range between about 660 nanometers (nm) and about 940 nm, and more particularly are wavelengths of about 761 nm, about 818 nm, about 665 nm, and about 894 nm.

Bearing the above in mind, according to certain embodiments of the present disclosure, each of the optical sensors is sensitive to light at a different respective wavelength, where each such wavelength is a wavelength at which light is efficiently reflected and/or transmitted by the septum of the animal. In such embodiments, the optical sensor 60a may be sensitive to light centered around approximately 761 nm, the optical sensor 60b may be sensitive to light centered around approximately 818 nm, the optical sensor 60c may be sensitive to light centered around approximately 665 nm, and the optical sensor 60d may be sensitive to light centered around approximately 894 nm. It is noted that these particular wavelengths are exemplary only, and optical sensors that are sensitive to other wavelengths, preferably in the range of 660 nm to 940 nm can also be used.

In certain embodiments, there is a correspondence between the number of light sources of the illumination arrangement 62 and the number of optical sensors. In particular, it may be preferable that there is a light source for each optical sensor, and that each light source is operative to produce light at the wavelength to which its corresponding optical sensor is sensitive. Thus, one of the light sources 64a may be operative to produce light centered around approximately 761 nm, another of the light sources 64b may be operative to produce light centered around 818nm, another of the light sources 64c may be operative to produce light centered around 665 nm, and another of the light sources 64d may be operative to produce light centered around 894 nm.

In the first set of embodiments (where the optical sensors operate to sense light reflected from the septum), the illumination arrangement 62 and the optical sensors are located on the same side of the septum. In such a deployment configuration, the illumination arrangement 62 and the optical sensors are located in the same arm member, and more preferably the illumination arrangement 62 is implemented as part of the optical module 58, such that the optical module 58 carries both the light source(s) 64 and optical sensors. In such an implementation, the light source(s) of the illumination arrangement 62 are located at the terminating end 26a of the animal engagement portion 22a of the arm member 16a together with the optical sensor(s).

In certain non-limiting implementations according to the first set of embodiments, the optical module 58 (having the optical sensors and the illumination arrangement) and the processor(s) 72b can be implemented using a commercial off-the-shelf integrated pulse oximeter and heart rate sensor which measures pulse rate and SpO₂. One such commercial off-the-shelf integrated pulse oximeter and heart rate sensor MAX30101, available from TinyCircuit, Akron, Ohio, USA, which is used extensively in wearable devices and smartphones.

In the second set of embodiments (where the optical sensors operate to sense light transmitted through the septum), the illumination arrangement 62 and the optical sensors are located on opposite sides of the septum. In such a deployment configuration, the illumination arrangement 62 and the optical sensors are located in different arm members. For example, the optical sensors 60a, 60b, 60c, 60d may be located at the terminating end 26a of the animal engagement portion 22a of the arm member 16a (as described above), and the illumination arrangement 62 may be located at the terminating end 26b of the animal engagement portion 22b of the arm member 16b. In such embodiments, the terminating end 26b preferably has an opening that can accommodate the illumination arrangement 62, and preferably also has a cover member with a light-transparent portion that covers the opening and allows the light generated by the illumination arrangement 62 to transmit therethrough to the septum.

In the deployment configuration in which the optical sensors operate to sense light transmitted through the septum, the terminating region 24b is an active region of the housing 12 in that it carries optical components (in this example the illumination arrangement 62). The terminating region 24b may also be made an active region by carrying sensor components, for example the thermal sensor 52. For example, the thermal sensor 52 can be carried by the animal engagement portion 22b of the arm member 16b (instead of the animal engagement portion 22a the arm member 16a as described in previous embodiments). In embodiments in which the thermal sensor is carried by the animal engagement portion 22b, a channel can be formed in at least part of the animal engagement portion 22b and at least part of the central portion 20b, similar to the channel 36 described in previous embodiments, to support a wired transmission medium that carries signal from the thermal sensor to the processing arrangement 70. If the processing arrangement 70 is located in the arm member 16a, the channel can be further extended through the base member 14 and other portions of the arm member 16a to allow the wired transmission medium to reach the processing arrangement 70.

With reference to FIG. 9, the processing arrangement 70 is a computerized processing unit that has one or more computerized (hardware) processors 72a and 72b coupled to one or more computerized storage medium 74a and 74b, such as a computerized memory or the like. As discussed above, the processing arrangement 70 is configured to process the signals received from the various sensors of the sensor arrangement 50 to calculate bioparameters of the animal, including breathing rate, pulse rate, and oxygen saturation level (SpO₂). The one or more processors 72a and 72b can be implemented as any number of computerized processors, including, but not limited to, microprocessors, microcontrollers, application-specific integrated circuits (ASICs), field programmable gate arrays (FPGAs), digital signal processors (DSPs), field-programmable logic arrays (FPLAs), and the like. In microprocessor implementations, the microprocessors can be, for example, conventional processors, such as those used in servers, computers, and other computerized devices. For example, the microprocessors may include x86 Processors from AMD and Intel, Xeon® and Pentium® processors from Intel, as well as any combinations thereof. The aforementioned computerized processors include, or may be in electronic communication with computer readable media, which stores program code or instruction sets that, when executed by the computerized processor, cause the computerized processor to perform actions. Types of computer readable media include, but are not limited to, electronic, optical, magnetic, or other storage or transmission devices capable of providing a computerized processor with computer readable instructions.

In certain embodiments, the processor(s) 72a is configured to perform processing functions on the signals received from the thermal sensor 52 to calculate breathing rate, and the the processor(s) 72b is configured to perform processing functions on the signals received from the optical sensors 60a, 60b, 60c, 60d to calculate pulse rate and oxygen saturation level (SpO₂). It is noted, however, that each of the processors 72a and 72b may be configured to calculate any of the aforementioned bioparameters. In particular, although the subdivision of the processing arrangement into separate elements 72a and 72b is shown in FIG. 9 according to a functional subdivision, it should be noted that these functions may be performed by processing arrangements which can be subdivided in any desired manner, with one or more function being performed by a single processor, or by a single function being performed by separate processors.

The storage medium 74a and 74b can be any conventional or application specific storage media, which although shown as single components for representative purposes, may each be multiple components. The storage medium 74a and 74b can be implemented in various ways, including, for example, one or more volatile or non-volatile memory, a flash memory, a read-only memory, a random-access memory, and the like, or any combination thereof. In certain embodiments, the storage medium 74a and 74b can include one or more components configured to store machine executable instructions that can be executed by the one or more processors 72a and 72b.

It is noted that although processing arrangement 70 is not shown in FIGS. 1-7, one or more of the processing elements of the processing arrangement 70 can be placed either externally to the housing 12 or internal to the housing 12. Thus, embodiments of the present disclosure provide options for local processing (i.e., processing within the housing 12), remote processing (i.e., processing external to the housing), and a combination thereof.

In certain embodiments, the processing element(s) of the processing arrangement 70 that process the signals from the thermal sensor 52 are external to the housing 12. In such embodiments, the wire(s) that carry the temperature-indicative signal from the body 55 of the thermal sensor 54 to the processing arrangement 70 protrude through the aperture or opening 40.

In certain embodiments, the processing element(s) of the processing arrangement 70 that process the signals from the optical sensors 60a, 60b, 60c, 60d are also external to the housing 12. In such embodiments, the wire(s) that carry the signals from the optical sensors 60a, 60b, 60c, 60d to the processing arrangement 70 may also protrude through the aperture or opening 40.

In certain preferred embodiments, the apparatus 10 is an untethered (i.e., wireless) device, whereby all of the processing elements of the processing arrangement 70 that process the signals from the sensors of the sensor arrangement 50 and the other electronic components (e.g., communications module 76 and power supply 78) are carried by the housing 12, and more preferably internal to the housing 12. In a particularly preferred but non-limiting implementation, the processing element(s) of the processing arrangement 70 that process the signals from the thermal sensor 52, and the processing element(s) of the processing arrangement 70 that process the signals from the optical sensors 60a, 60b, 60c, 60d, are placed in an interior portion of the housing 12, for example in the channel 36 or another hollowed section of the housing 12. In such embodiments, the aperture or opening 40 is not needed, as all of the processing components are internal to the housing 12. In such embodiments, the processing arrangement 70, the communications module 76, and the power supply 78 are of minimal size so as to be able to be placed within the housing 12, for example in the channel 36.

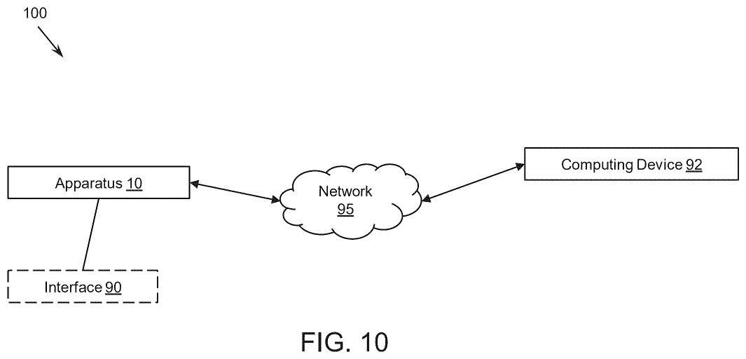

With continued reference to FIGS. 1-9, refer now to FIG. 10 which shows an example of a system 100 according to a non-limiting embodiment of the present disclosure. The system 100 includes the apparatus 10, which itself includes the processing arrangement 70, and a computing device 92 that is remotely located form the animal and the apparatus 10. The computing device 92 can be any computer or computer system having one or more hardware processors coupled to one or more computerized storage medium (e.g., memory). Examples of computers and computer systems that can be used to implement the computing device 92 include, but are not limited to, desktop computers, laptop computers, tablet computers, smartphones, and server computers.

In certain embodiments, the bioparameter data that is calculated by the processing arrangement 70 can be sent to the computing device 92 for further processing, for example statistical processing or other type of processing to extract diagnostic or other information from the bioparameters. In certain embodiments, the computing device 92 may also receive the raw sensor measurements from one or more of the sensors of the sensor arrangement 50. For example, the apparatus 10 may send the raw thermal sensor measurements to the computing device 92, and to extract information associated with the animal including at least diagnostic information. For example, the computing device 92 may process the raw thermal sensor measurements using anomaly detection or other algorithms to detect critical conditions in the animal.

In order to transmit the bioparameters and/or the raw sensor measurements to the computing device 92, the apparatus 10 preferably includes a communications module 76 that is configured to transmits data over a wireless communication network 95, which can be formed from one or more networks, including, for example, the Internet, cellular networks, local area networks, and personal area networks. The communications module 76 includes one or more transceiver devices capable of transmitting and receiving communication signals and data over a communication channel (e.g., network). For example, if the communications module 76 is implemented as a cellular module, the cellular module can include a MODEM/transceiver that exchanges signals and data according to any suitable cellular communication protocol, including, for example, 4G/LTE, 5G, GSM, and the like. As an additional example, if the communications module 76 is implemented as a wireless module, the wireless module can include a MODEM/transceiver that supports communication using wireless local area network (WLAN) protocols (commonly referred to as Wi-Fi) and/or a MODEM/transceiver that supports Bluetooth communication protocols.

It is noted that in embodiments in which the processing arrangement 70 is external to the housing 12, the computing device 92 can be incorporated as part of the processing arrangement.

In certain embodiments, the system 100 can also include an interface 90, for example, a hand-held push button, that can be connected or linked to the apparatus 10, for example via the processing arrangement 70. The interface 90 is provided to allow a user of the apparatus 10, for example a veterinarian, to provide data input while the user physically monitors the animal. In certain embodiments, the interface 90 is linked to the apparatus 10 (processing arrangement 70) by a wired data connection. In other embodiments, the interface 90 is linked to the computing device 92 by a wired data connection, which provides a wireless connection between the interface 90 and the processing arrangement 70.

In use, for example, the user can actuate the interface 90, for example by pressing the push-button, each time the animal performs a certain movement, such as a flank movements. Flank movement counting is a conventional method for measuring animal breathing rate. The flank movements can be recorded by the processing arrangement 70 and/or the computing device 92, and correlated with the bioparameters that are calculated by the processing arrangement 70. For example, the user interface 90 can be used to count the flank movements, and the computing device 92 and/or the processing arrangement 70 can compare the counted flank movements with the breathing rate calculated from the thermal sensor measurements.

Although the embodiments described herein have pertained to a sensor arrangement having both a thermal sensor (or multiple thermal sensors) for collecting measurements used for calculating breathing rate and one or more optical sensors for collecting measurements used for calculating pulse rate and oxygen saturation, embodiments of the disclosed apparatus are contemplated in which the sensor arrangement includes only thermal sensors for calculating breathing, and embodiments of the disclosed apparatus are contemplated in which the sensor arrangement includes only optical sensors for calculating pulse rate and oxygen saturation.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein, the singular form, “a”, “an” and “the” include plural references unless the context clearly dictates otherwise.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements. To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the disclosure.

To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the disclosure.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. An apparatus for monitoring an animal, the apparatus comprising:
a housing configured to engage with nasal cavities of the animal, the housing including a first arm member and a second arm member, each of the arm members having an animal engagement portion configured to be inserted into one of the nasal cavities;
a sensor arrangement carried by the housing and including:
a thermal sensor having at least a portion thereof located in the animal engagement portion of the first or second arm member, the thermal sensor for measuring nasal cavity air temperature, and
at least one optical sensor located at a terminating end of the animal engagement portion of the first or second arm member, the at least one optical sensor for sensing light from the septum of the animal; and
a processing arrangement in communication with the housing and electrically associated with the sensor arrangement, the processing arrangement configured to:
process signals from the thermal sensor to calculate at least one respiration-related bioparameter of the animal, and
process signals from the at least one optical sensor to calculate at least one blood circulation-related bioparameter of the animal.

2. The apparatus of claim 1, wherein the at least one optical sensor senses light, produced by at least one light source, that is reflected from the septum.

3. The apparatus of claim 1, wherein the at least one optical sensor senses light, produced by at least one light source, that is transmitted by the septum.

4. The apparatus of claim 1, further comprising: an illumination arrangement including at least one light source for producing the light from the septum of the animal that is sensed by the at least one optical sensor, wherein the illumination arrangement is located at a terminating end of the animal engagement portion of the first arm member.

5. The apparatus of claim 1, further comprising: an illumination arrangement including at least one light source for producing the light from the septum of the animal that is sensed by the at least one optical sensor, wherein the illumination arrangement is located at a terminating end of the animal engagement portion of the second arm member.

6. The apparatus of claim 1, wherein the at least one optical sensor includes a plurality of optical sensors, each of the optical sensors being sensitive to a different respective wavelength.

7. The apparatus of claim 1, wherein the thermal sensor and the at least one optical sensor are located in the same arm member.

8. The apparatus of claim 1, wherein the thermal sensor and the at least one optical sensor are located in different arm members.

9. The apparatus of claim 1, wherein the first or second arm member has a hollow interior section forming a channel in at least part of the animal engagement portion, wherein an aperture in a major external surface of the animal engagement portion communicates with the channel, and wherein a portion of the thermal sensor is located in the channel and another portion of the thermal sensor protrudes from the aperture.

10. The apparatus of claim 1, wherein the first or second arm member has a hollow interior section forming a channel in at least part of the animal engagement portion and at least part of a central portion of the first or second arm member, and wherein at least part of one or more wired transmission medium that carry signal from one or more sensor of the sensor arrangement to the processing arrangement is located in the channel.

11. The apparatus of claim 1, wherein each of the animal engagement portions includes a terminating end, and wherein one of the terminating ends is circular shaped to reduce discomfort to the animal.

12. The apparatus of claim 1, wherein each of the animal engagement portions includes a terminating end, and wherein one of the terminating ends has a configuration that accommodates an optical module having the at least one optical sensor and that reduces discomfort to the animal.

13. The apparatus of claim 1, wherein each of the animal engagement portions includes a terminating end, and wherein the housing includes an adjustment mechanism configured to adjust a lateral spacing between the terminating ends.

14. The apparatus of claim 1, the housing further including a base member, and each of the arm members includes a base engagement portion that mechanically couples the arm member to the base member, at least one of the base engagement portions configured to slide along the base so as to adjust a lateral spacing between the terminating ends.

15. The apparatus of claim 1, wherein the processing arrangement includes at least one first processor for processing signals from the thermal sensor and at least one second processor for processing signals from the at least one optical sensor.

16. The apparatus of claim 1, wherein at least one processor of the processing arrangement is carried by the housing.

17. The apparatus of claim 1, wherein the processing arrangement is internal to the housing.

18. The apparatus of claim 1, further comprising: a communication module configured to send one or more of the calculated bioparameters to a computing device that is remote from the apparatus.

19. The apparatus of claim 18, wherein the communication module is further configured to send to the computing device raw sensor measurements from one or more of the thermal sensor or the at least one optical sensor, and wherein the computing device is configured to process the raw sensor measurement to extract information associated with the animal including at least diagnostic information.

20. An apparatus for monitoring an animal, the apparatus comprising:
a housing configured to engage with the nasal cavities of the animal, the housing including:
a first arm member and a second arm member, each of the arm members having an animal engagement portion configured to be inserted into one of the nasal cavities, each of the animal engagement portions including a terminating end, and
an adjustment mechanism configured to adjust a lateral spacing between the terminating ends; and
a sensor arrangement carried by the housing and including one or more sensor located in one of the animal engagement portions and operative to collect sensor measurements from one of the nasal cavities indicative of one or more bioparameter of the animal.

21. The apparatus of claim 20, further comprising: a processing arrangement carried by the housing, the processing arrangement including at least one hardware processor in communication with at least one storage medium and configured to process signals from the one or more sensor to derive the one or more bioparameter.

22. A system for monitoring an animal, the system comprising:
an animal monitoring apparatus including:
a housing configured to engage with the nasal cavities of the animal, the housing including a first arm member and a second arm member, each of the arm members having an animal engagement portion configured to be inserted into one of the nasal cavities,
a sensor arrangement carried by the housing and including:
a thermal sensor having at least a portion thereof located in the animal engagement portion of the first or second arm member, the thermal sensor for measuring nasal cavity air temperature, and
at least one optical sensor located at a terminating end of the animal engagement portion of the first or second arm member, the at least one optical sensor for sensing light from the septum of the animal, and
a communications module carried by the housing;
a processing subsystem in communication with the sensor arrangement and configured to:
process signals from the thermal sensor to calculate at least one respiration-related bioparameter of the animal, and
process signals from the at least one optical sensor to calculate at least one blood circulation-related bioparameter of the animal; and
a computing device that is in communication with the animal monitoring apparatus via one or more wireless communication network,
wherein the communications module is configured to send to the computing device, over the one or more wireless communication network, raw sensor measurements from the sensor arrangement, and
wherein the computing device is configured to process the raw sensor measurement to extract information associated with the animal including at least diagnostic information.

23. The system of claim 22, wherein the animal monitoring apparatus includes the processing subsystem, and wherein the processing subsystem is carried by the housing.
